(19) United States
(12) Patent Application Publication
(10) Pub. No.: US 2003/0131489 A1
(43) Pub. Date: Jul. 17, 2003

(54) Measuring instrument for measuring the girth of fingers

(76) Inventor: Chih Ming Hsiao, Wur Shiang (TW)

Correspondence Address: PRO-TECHTOR INTERNATIONAL SERVICES, 20775 Norada Court, Saratoga, CA, 95070-3018, US

(21) Appl. No.: 10/052,073

(22) Filed: Jan. 16, 2002

(51) Int. Cl. G01B 3/34; A61B 5/107

(52) U.S. Cl. 33/555.2; 33/514.1

(57) ABSTRACT

A measuring instrument for measuring the girth of fingers is constructed to include at least one measuring card, each measuring card having a set of finger holes of different diameters for the insertion of fingers and markings indicative of the diameters of the finger holes respectively, and at least one reference card respectively pivoted to the at least one measuring card, each reference card having printed thereon advertising matters.

### BACKGROUND OF THE INVENTION

1. Field of the Invention

The present invention relates to a measuring instrument and, more specifically, to an instrument for measuring the girth of fingers.

2. Description of the Related Art

A finger girth measuring instrument is known comprised of a string of metal rings of different diameters. This finger girth measuring instrument is heavy and inconvenient in use. Furthermore, different molds should be used for making metal rings of different diameters, the manufacturing cost of this design of finger girth measuring instrument is high.

### SUMMARY OF THE INVENTION

The present invention has been accomplished to provide a measuring instrument for measuring the girth of fingers, which eliminates the aforesaid drawbacks. It is one object of the present invention to provide a measuring instrument for measuring the girth of fingers, which is easy to use. It is another object of the present invention to provide a measuring instrument for measuring the girth of fingers, which is inexpensive to manufacture. It is still another object of the present invention to provide a measuring instrument for measuring the gird of fingers, which can be used as advertising means for business promotion. According to one aspect of the present invention, the measuring instrument is comprised of at least one measuring card with finger holes for measuring the girth of fingers, and at least one reference card for advertising purpose. According to another aspect of the present invention, the cards are respectively made of cheap sheet material and pivotally connected together.

### BRIEF DESCRIPTION OF THE DRAWINGS

FIG. 1 is an elevational view of a measuring card for a measuring instrument according to the present invention.

FIG. 2 is an elevational view of a reference card for a measuring instrument according to the present invention.

FIG. 3 is a schematic drawing showing the application of a measuring instrument constructed according to one embodiment of the present invention.

FIG. 4 is a schematic drawing showing the application of a measuring instrument constructed according to a second embodiment of the present invention.

FIG. 5 is a schematic drawing showing the application of a measuring instrument constructed according to a third embodiment of the present invention.

### DETAILED DESCRIPTION OF THE PREFERRED EMBODIMENT

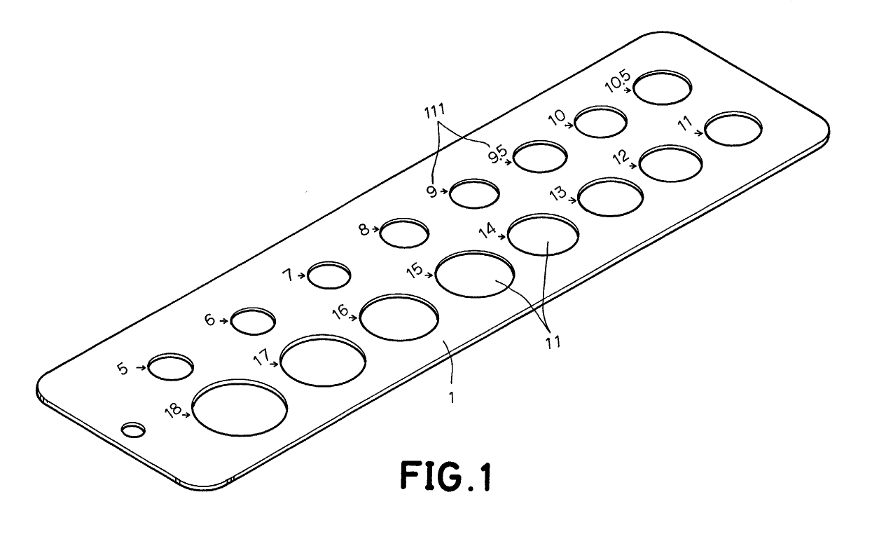

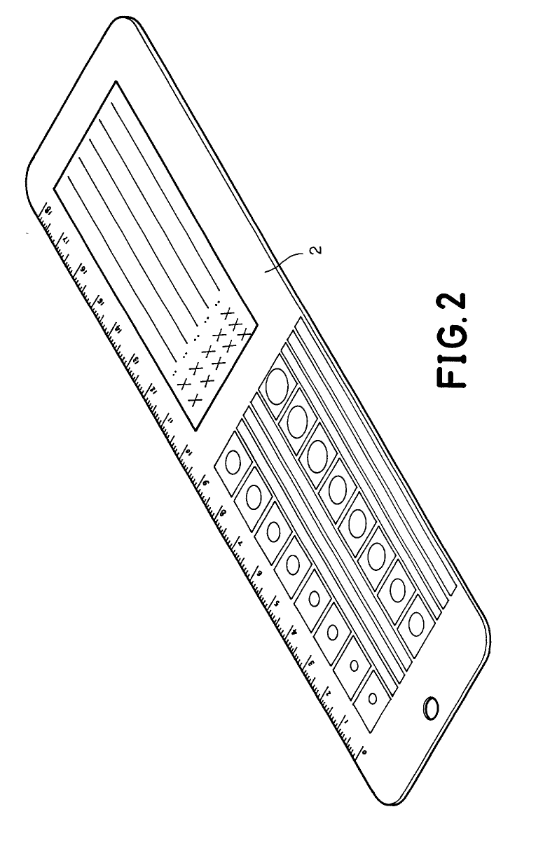

Referring to FIGS. 1 and 2, a measuring instrument for measuring the girth of fingers in accordance with one embodiment of the present invention is generally comprised of at least one measuring card 1, and at least one reference card 2.

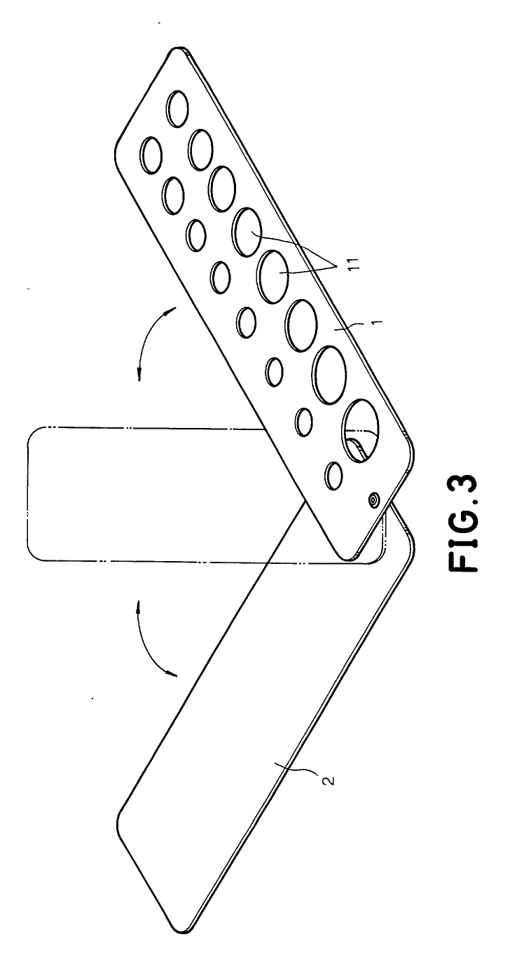

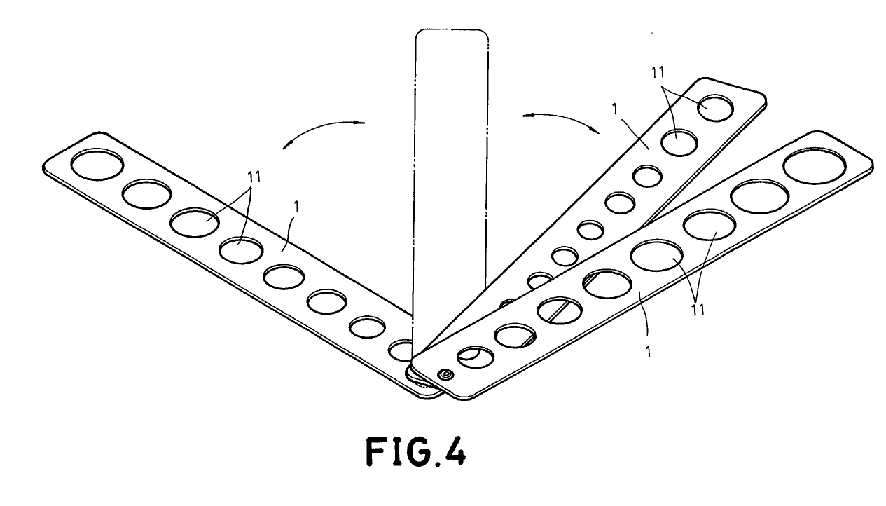

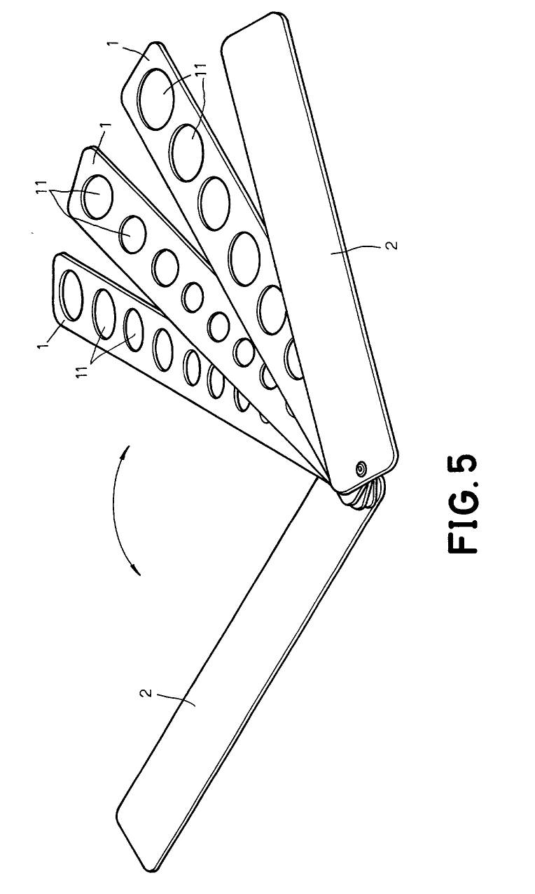

Referring to FIG. 1 again, the measuring cards 1 are planar plate members, each comprising a set of finger holes 11 of different diameters, and markings 111 respectively disposed corresponding to the finger holes 11 to indicate the sizes of the finger holes 11 respectively. The finger holes 11 may be arranged in one line as shown on FIGS. 4 and 5, or two lines as shown on FIGS. 1 and 3. When in use, the user attempts inserting the finger into the finger holes 11 selectively to see which finger hole 11 fits the finger perfectly. From the indication of the marking 111 corresponding to the finger hole 11 that fits the finger perfectly, the user knows the size of the finger measured.

Referring to FIG. 2 again, the reference cards 2 are planar plate members respectively printed with a calendar, conversion table, measuring rule, trademark, logo, advertising patterns or words, and etc.

Referring to FIGS. from 3 through 5, the measuring cards 1 and the reference cards 2 are pivotally connected together so that the cards 1 and 2 can be spread out or received. Alternatively, the cards 1 and 2 can be hinged together like the binding of a book. According to the example shown on FIG. 3, one reference card 2 is provided at the bottom side, serving as a back cover. According to the example shown on FIG. 5, two reference cards 2 are provided at the top and bottom sides, serving as a front cover and a back cover respectively.

The aforesaid measuring cards 1 and reference cards 2 can be made of paper cards, plastic cards, metal plates, wooden plates, etc. The measuring instrument of the present invention can be used as a small gift for the consumers to promote the sales.

A prototype of measuring instrument for measuring the girth of fingers has been constructed with the features of FIGS. 1˜5. The measuring instrument for measuring the girth of fingers functions smoothly to provide all of the features discussed earlier.

Although particular embodiments of the invention have been described in detail for purposes of illustration, various modifications and enhancements may be made without departing from the spirit and scope of the invention. Accordingly, the invention is not to be limited except as by the appended claims.

What the invention claimed is:

1. A measuring instrument for measuring the girth of fingers, comprising at least one measuring card, said measuring card comprising a set of finger holes of different diameters for the insertion of fingers, and markings indicative of the diameters of said finger holes respectively.

2. The measuring instrument for measuring the girth of fingers as claimed in claim 1, further comprising at least one reference card respectively pivoted to said at least one measuring card, said at least one reference card having printed thereon a measuring rule, a calendar, a conversion table, and advertising matters.

3. The measuring instrument for measuring the girth of fingers as claimed in claim 1, wherein said at least one measuring card and said at least one reference card are respectively made of paper.

4. The measuring instrument for measuring the girth of fingers as claimed in claim 1, wherein said at least one measuring card and said at least one reference card are respectively made of plastics.

5. The measuring instrument for measuring the girth of fingers as claimed in claim 1, wherein said at least one measuring card and said at least one reference card are respectively made of metal.

6. The measuring instrument for measuring the girth of fingers as claimed in claim 1, wherein said at least one measuring card and said at least one reference card are respectively made of metal.
